(19) United States
(12) Patent Application Publication
(10) Pub. No.: US 2026/0285811 A1
(43) Pub. Date: Sep. 24, 2026

(54) PROCESS FOR THE PRODUCTION OF 4-AMINO-5-METHYL-(1H)-PYRIDIN-2-ONE

(71) Applicant: MINASCENT TECHNOLOGIES GMBH, LEUNA (DE)

(72) Inventors: Martin KÜHNAST, HALLE (SAALE) (DE); Jean NONNENMACHER, MERSEBURG (DE); Yvonne RICHTER, MÜCHELN (GEISELTAL) (DE)

(73) Assignee: MINASCENT TECHNOLOGIES GMBH, LEUNA (DE)

(21) Appl. No.: 19/471,870

(22) PCT Filed: Mar. 11, 2024
(86) PCT No.: PCT/EP2024/056404

§ 371 Date: Oct. 2, 2025

(30) Foreign Application Priority Data
Apr. 5, 2023 (EP) 23166713.0

(51) Int. Cl. C07D 213/73

(52) U.S. Cl. CPC C07D 213/73

(57) ABSTRACT

An improved process for the production of 4-amino-5-methyl-(1H)-pyridin-2-one (formula II). The process includes the reaction of 4-hydroxy-5-methyl-(1H)-pyridin-2-one (formula (I)) under pressure with aqueous ammonia and a salt or an acid to provide the target compound in high yield and high purity. The pressure may be an externally applied pressure of 5 to 340 bar.

### TECHNICAL FIELD

The instant application concerns an improved process for the production of 4-amino-5-methyl-(1H)-pyridin-2-one (formula II):

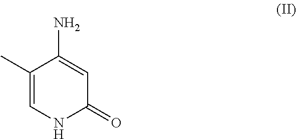

### BACKGROUND AND PRIOR ART

The compound of formula II is a key intermediate for the synthesis of finerenone:

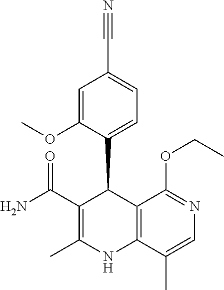 [SMILES: CCOc1ncc(C)c2c1[C@H](c1ccc(C#N)cc1OC)C(C(N)=O)=C(C)N2]

Finerenone has efficacy as non-steroidal mineralocorticoid receptor antagonist (MRA). It is used as a medicament for the prophylaxis and/or treatment of cardiovascular or renal diseases.

The compound of formula II is also an intermediate for the synthesis of omeprazole-derivatives as disclosed in CN103193704 A.

A synthesis of the 4-amino-5-methyl-(1H)-pyridin-2-one (formula II) has been published in the journal Synthesis, 1984, volume 9, pages 765 to 766, see example 3c. In a first step 4-hydroxy-5-methyl-(1H)-pyridin-2-one (formula I) is reacted with benzylamine under reflux, thereby substituting the hydroxy group by the benzylamine group. In a second step the benzylamine group is cleaved off by way of catalytic hydrogenation over palladium on charcoal. The total yield over both steps is 62.4%:

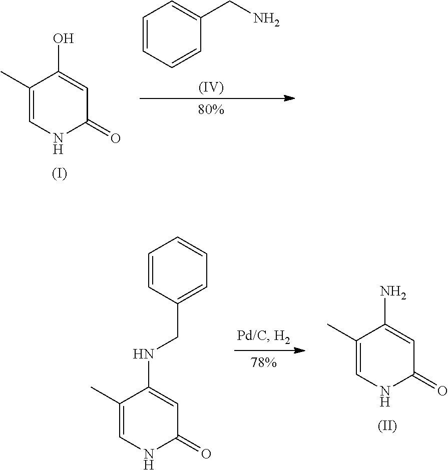

The synthesis is disadvantageous in that a massive excess (ca. 9-fold) of benzylamine is necessary. The recycling of excessive benzylamine is complicated and costly. The reaction requires a temperature of 185 degrees Celsius to achieve boiling and reflux temperatures; and it takes 36 hours. Such high temperatures cannot be achieved in standard reflux reactors. Therefore, special equipment is necessary.

Moreover, it has been reported in WO 2020/178177 A1 that the following side product is formed during the first step of the synthesis in non-negligible concentration at large scale:

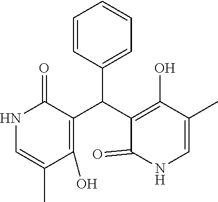 [SMILES: Cc1c[nH]c(=O)c(C(c2ccccc2)c2c(O)c(C)c[nH]c2=O)c1O]

This compound is separated from the target compound using chlorinated solvents, which are preferably to be avoided. The hydrogenation requires enormous volumes of solvents and palladium on charcoal. The work-up of the hydrogenation reaction requires a step of chromatography. Thus, the synthesis is economically not viable for large scale production.

Reactions of phenols with ammonia are described in literature: EP 2543654 A1, 2013 (Sumitomo Rubber Industries, Ltd) discloses the reaction of phenol with ammonia at 450° C. (21.2% yield); I. G. Farbenind. Patent: DE570365, 1930; Fortschr. Teerfarbenfabr. Verw. Industriezweige, vol. 18, p. 446 und Fischer; Bahr; Wiedeking Brennstoff-Chemie, 1934, vol. 15, p. 101, 103 describe similar processes under similarly drastic conditions. See also Chem. News J. Ind. Sci., 1867, vol. 16, p. 55; Helv. chim. Acta, 1924, vol. 7, p. 282; Gmelin Handbook: N: MVol. 2, 5.2.2, page 490-493.

Reactions involving naphthalene are described in WO2008/124812 A1, 2008 and in Chemistry—An Asian Journal, 2010, vol. 5, #9 p. 2053-2061 and US 2008/293766 A1, 2008 and EP 2075245 A2, 2009 and Synthetic Communications, 2001, vol. 31, #14 p. 2143.

These are special variants of the so-called Bucherer-reaction and cannot be applied to 4-amino-5-methyl-(1H)-pyridin-2-one (formula II).

According to WO 2020/178177 A1, the processes of the prior art involve rather drastic reaction conditions like temperatures of above 300° C., while only achieving rather low yields. Processes that involve the addition of a sulfite and Bucherer-type reactions, respectively, are not applicable for the conversion of compound (I) to compound (II). Moreover, an attempt at a direct conversion of compound (I) to compound (II), wherein compound (I) was exposed to gaseous ammonia in an autoclave at temperatures up to 180° C. afforded the target compound (II) in only minor amounts.

A one-step conversion of compound (I) to compound (II) is described in WO 2020/178177 A1 (Bayer AG). The publication discloses that an amino group can be introduced into compound (I) by reacting compound (I) with ammonia under addition of an ammonium bromide salt in an autoclave (high pressure reactor) thus affording compound (II) at a yield of more than 90%. It is disclosed that 0.2 to 3 equivalents, preferably one equivalent, of an ammonium bromide, such as ammonium bromide or tri-alkyl-ammonium bromide or a tetra-alkyl-ammonium bromide, preferably ammonium bromide, are reacted with compound (I) in an autoclave, into which ammonia is fed by condensation to function as reagent and solvent (40 to 100 equivalents, preferably 40 to 60 equivalents, with examples having 50 equivalents of ammonia). WO 2020/178177 A1 discloses reaction temperatures of 150 to 200° C., particularly preferred to 170° C., and pressure in the autoclave goes up to 70 to 90 bar as per examples.

Advantages of the process over the prior art, in particular the process described in the article published in the journal Synthesis, 1984, volume 9, pages 765 to 766 discussed above, are as follows: only one step of synthesis is necessary, higher yields are achieved, a catalyst such as palladium on charcoal is not necessary, chromatographic purification of the raw-product is unnecessary (the target compound (II) is obtained directly in high purity as a crystalline product), excessive use of organic solvents, in particular chlorinated solvents, is unnecessary, and high reaction temperatures need not be attained.

However, the process disclosed in WO 2020/178177 A1 suffers from the disadvantage that large amounts of ammonia are necessary due to its use as solvent. This requires special equipment to set up the reaction, for safe removal of ammonia after completion of the reaction as well as for recycling of ammonia. Also, after removal of ammonia a dry powder is obtained, which is more difficult to handle and discharge from a large reactor than a solution or slurry.

Therefore, there is a need to provide an alternative synthesis of 4-amino-5-methyl-(1H)-pyridin-2-one (formula II), which is suitable for industrial production, avoids excessive use of solvents and reagents, in particular ammonia, and can be carried out with standard equipment.

### SUMMARY

The present disclosure provides a process for the production of 4-amino-5-methyl-(1H)-pyridin-2-one of formula (II)

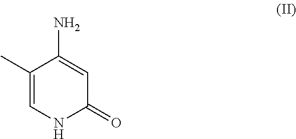

characterised in that 4-hydroxy-5-methyl-(1H)-pyridin-2-one of formula (I)

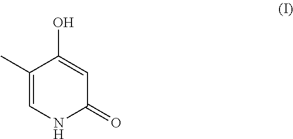

is reacted under pressure with aqueous ammonia and a salt or an acid.

The process of the present disclosure overcomes the above disadvantages. The process has the advantages that ammonia as solvent has become obsolete, and the reaction can be run in standard solvents while employing a significantly smaller number of equivalents of ammonia than in WO 2020/178177 A1. Consequently, special equipment for the handling of vast amounts of gaseous ammonia, its removal after reaction and its recycling are not required. Very high reaction temperatures are unnecessary. Vast volumes of solvents are neither required for the reaction nor for the work-up of the crude product. The work-up of the reaction is easier to carry out using standard equipment to discharge a slurry from the reactor followed by filtering of the slurry. The process of the present disclosure affords compound (II) in high to very high yields. Therefore, the process is suitable for industrial scale-up.

### DETAILED DESCRIPTION

Further aspects, features and advantages of the exemplary embodiments will become apparent from the detailed description which follows.

The patents, published applications and scientific literature referred to herein establish the knowledge of those with skill in the art and are hereby incorporated by reference in their entireties to the same extent as if each was specifically and individually indicated to be incorporated by reference.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a method, the term “comprising” means that the method includes at least the recited steps, but may include additional steps.

The terms “consists essentially of” or “consisting essentially of” have a partially closed meaning, that is, they do not permit inclusion of steps or features or components which would substantially change the essential characteristics of a method or composition; for example, steps or features or components which would significantly interfere with the desired properties of the compounds or compositions described herein, i.e., the method or composition is limited to the specified steps or materials and those which do not materially affect the basic and novel characteristics of the method or composition. The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth.

The term “dissolved” or “substantially dissolved” is used herein to mean the solubilization of a solid in a solution. It can be considered that a solid is “dissolved” or “substantially dissolved” in a solution when the resulting solution is clear or substantially clear.

As used herein, the recitation of a numerical range for a variable is intended to convey that the variable can be equal to any values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise.

Technical and scientific terms used herein have the meaning commonly understood by one of skill in the art to which the present description pertains, unless otherwise defined.

The process of the present disclosure is characterized in that 4-hydroxy-5-methyl-(1H)-pyridin-2-one of formula (I)

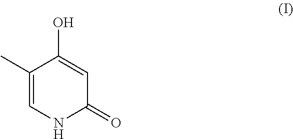

is reacted under pressure with aqueous ammonia and a salt or an acid to afford 4-amino-5-methyl-(1H)-pyridin-2-one of formula (II)

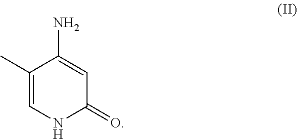

The starting material, 4-hydroxy-5-methyl-(1H)-pyridin-2-one (formula (I)), is commercially available or may be synthesised according to the method described in the article in Synthesis, 1984, volume 9, pages 765 to 766 discussed above.

The reaction is preferably carried out at 120 to 220° C., 140 to 200° C., preferably 150 to 190° C., more preferably 165 to 175° C. The reaction may be carried out under stirring or without stirring, preferably under stirring.

The reaction is carried out under pressure. The reaction may be carried out in an autoclave under autogenous pressure, i.e. the pressure in the autoclave is generated by heating to achieve above reaction temperatures and is dependent on the volume of the autoclave. The pressure in the autoclave may be at least 5, 10, 20, 25, or 30 bar and up to 100, 110, 200 or even 340 bar. The pressure may preferably be 15 to 100 bar, more preferably 20 to 100 bar. The reaction may alternatively be carried out in a pressure reactor by applying pressure externally, for example by exposing the reaction mixture to a gas pressure. The pressure in the pressure reactor may be at least 10, 20, 25, or 30 bar and up to 100, 110, 200 or even 340 bar. The pressure may preferably be 15 to 100 bar, more preferably 20 to 100 bar. The reaction may alternatively be carried out in continuous mode using a tubular reactor, wherein aforesaid pressure ranges may be applied on a solution by a pump effecting compression of a solution.

The process requires the presence of aqueous ammonia. Ammonia may be present in the reaction solution in an amount of 10 to 20 molar equivalents based on formula (I), preferably 13 to 17 molar equivalents, and more preferably 14 to 16 molar equivalents. In an embodiment commercially available aqueous ammonia solution (25%) may be used. Alternatively, aqueous ammonia solutions having 5 to 30% may be used. At proportions of lower than 5% the reaction becomes unproductive. At proportions much higher than 30% solutions are unstable due to degassing of ammonia under atmospheric pressure at room temperature. Using an aqueous ammonia solution, a significantly smaller number of equivalents of ammonia than in the WO 2020/178177 A1 can be used. Handling of gaseous ammonia is no longer necessary. Special equipment for the handling of vast amounts of gaseous ammonia before and after reaction are no longer required.

The process is preferably carried out in the presence of a salt. A salt used in the process may be an inorganic salt or a salt that comprises organic substituents. Suitable salts include salts based on cations selected from alkali metals or comprising a nitrogen-based cation. Suitable counter-ions may be selected from halides, preferably fluoride, chloride, bromide and iodide, as well as sulfate, sulfite, phosphates, preferably phosphate tribasic, and organic anions such as carboxylates, e.g. acetate, formate, citrate, oxalate, tartrate, and sulfonates, preferably mesylate, tosylate, triflate.

In one embodiment, the salt has the formula of [NR₄]ₓM, wherein R stands for a C1 to C6 alkyl group or hydrogen, M for an inorganic or organic anion and x corresponds to the number of charge of the anion. In one embodiment the salt comprises a nitrogen-based cation which may be selected from the group consisting of ammonium, mono-alkyl-ammonium, di-alkyl-ammonium, tri-alkyl-ammonium, tetra-alkyl-ammonium, pyridinium, and lutidinium, and an anion selected from the group consisting of fluoride, chloride, bromide, iodide, sulfate, sulfite, triflate, phosphate tribasic, acetate, citrate, formate, oxalate, tartrate, mesylate, triflate and tosylate. Preferably, the nitrogen-based cation is selected from the group consisting of ammonium, mono-alkyl-ammonium, di-alkyl-ammonium, tri-alkyl-ammonium and tetra-alkyl-ammonium, and the anion is selected from fluoride, chloride, bromide, iodide, sulfate, sulfite, phosphate tribasic, acetate, citrate, formate, oxalate, tartrate, mesylate, triflate and tosylate. The salt may be added in 0.2 to 15 molar equivalents based on formula (I), wherein the molar equivalents are calculated with respect to the ammonium cation of a salt. In an example, a mono-ammonium salt is added in 0.2 to 15 molar equivalents based on formula (I). A bis-ammonium salt is added in 0.1 to 7.5 molar equivalents based on formula (I). The alkyl group in the mono-alkyl-ammonium, di-alkyl-ammonium, tri-alkyl-ammonium and tetra-alkyl-ammonium cations may be a C1 to C6 alkyl group, preferably a methyl, ethyl, propyl or butyl group.

The salt may be selected from the group consisting of ammonium, mono-alkyl-ammonium, di-alkyl-ammonium, tri-alkyl-ammonium, or tetra-alkyl-ammonium fluoride; ammonium, mono-alkyl-ammonium, di-alkyl-ammonium, tri-alkyl-ammonium, or tetra-alkyl-ammonium chloride; ammonium, mono-alkyl-ammonium, di-alkyl-ammonium, tri-alkyl-ammonium, or tetra-alkyl-ammonium bromide; and ammonium, mono-alkyl-ammonium, di-alkyl-ammonium, tri-alkyl-ammonium, tetra-alkyl-ammonium iodide, ammonium triflate, ammonium phosphate, ammonium acetate, ammonium citrate, ammonium oxalate, ammonium tartrate, ammonium mesylate or ammonium tosylate, pyridinium bromide. The salt may preferably be ammonium chloride. Aforesaid specific salts may be added in 5 to 15 or 5 to 10 molar equivalents based on formula (I), preferably in an amount of 6 to 8 molar equivalents based on formula (I), e.g. about 7 molar equivalents, wherein the molar equivalents are calculated with respect to the ammonium cation of a salt.

The salt may be selected from the group consisting of ammonium, mono-alkyl-ammonium, di-alkyl-ammonium, tri-alkyl-ammonium, or tetra-alkyl-ammonium sulfate; and ammonium, mono-alkyl-ammonium, di-alkyl-ammonium, tri-alkyl-ammonium, or tetra-alkyl-ammonium sulfite. Said salts may be added in an amount of 0.1 to 6, 0.1 to 5, 0.2 to 4.0, 0.2 to 3.5 or 0.5 to 3.0 molar equivalents based on formula (I), wherein the molar equivalents are calculated with respect to the ammonium cation of a salt.

The salt used in the process may be formed in situ by an addition of a corresponding base of the cations described in the paragraph above and a corresponding acid of the anions described in the paragraph above in required respective molar ratios. The corresponding bases and corresponding acids may be added in the molar equivalents based on formula (I) as described in the three paragraphs above.

In a different embodiment, a salt may be added which does not comprise a nitrogen-based cation such as an ammonium cation. Such a salt may be selected from alkali metal halides and earth alkali metal halides, in particular from the group consisting of alkali metal fluoride, chloride, bromide, iodide, tosylate, triflate; and alkaline earth metal fluoride, chloride, bromide, or iodide, tosylate, triflate or carbonate. Alkali metals may comprise lithium, sodium or potassium. The alkaline earth metals may comprise magnesium, calcium, strontium and barium. Sodium iodide and potassium iodide are preferred. These salts may be added in an amount of 0.25 to 9, 0.5 to 8, 0.75 to 7 or 1 to 6 molar equivalents based on formula (I).

In another embodiment, the reaction as disclosed in the above paragraphs is carried out by additionally adding a Lewis acid catalyst. In some embodiments the Lewis acid catalyst may be added instead of the salt which does not comprise a nitrogen-based cation such as an ammonium cation as described in the preceding paragraph. A salt of a metal such as magnesium, calcium, aluminium, boron, silicon, zinc, iron, copper, silver, titanium and zirconium may be added to the reaction. The counter ion of the salt may be an inorganic anion such as halides, preferably fluoride, chloride, bromide and iodide, as well as sulfate, sulfite, carbonate, phosphates, preferably phosphate tribasic, and organic anions such as carboxylates, e.g. acetate, formate, citrate, and sulfonates, preferably mesylate, tosylate, triflate. As Lewis acid catalyst zinc acetate, aluminium lactate, magnesium chloride, iron acetate, copper sulfate, zinc chloride, or calcium chloride may be used. The Lewis acid catalyst may be added in the form of a powder. The proportion of the catalyst may be 2 to 20 mol %, or 5 to 15 mol %, or 7 to 13 mol %, or 8 to 12 mol %, or 3 to 8 mol % with regard to formula (I).

The proportions of aqueous ammonia and ammonium salt may be as described above. In an example, aqueous ammonia, ammonium bromide and aluminium lactate are reacted. In another example, aqueous ammonia, ammonium bromide and magnesium chloride are reacted.

The temperatures applied in the reaction may be as described above. However, they can be as low as 130 to 170° C., 140 to 160° C., or 145 to 155° C., preferably 150° C. The pressure of the reaction may be as described above. But it can be as low as 5 bar.

The advantage of the addition of a Lewis acid catalyst is that the reaction proceeds at low temperatures of 150° C. and gives high yields. It also proceeds at lower pressures.

In another embodiment, the reaction as disclosed herein is carried out in the presence of an acid instead of the salt described above. The addition of an acid in the presence of ammonia affords an in-situ generation of an ammonium salt. The acid may be selected from an organic or inorganic acid that shows at least moderate solubility in water. The acid may be added in an amount of 3 to 10, preferably 4 to 8 molar equivalents based on formula (I). The organic acid may be a carboxylic acid or a sulfonic acid. The organic acid may be selected from citric acid, oxalic acid, tartaric acid, lactic acid, malonic acid, caproic acid, formic acid, acetic acid, propionic acid, butanoic acid, isobutanoic acid, pentanoic acid, trifluoroacetic acid, methanesulfonic acid, para-toluenesulfonic acid, and trifluoromethanesulfonic acid. The inorganic acid is selected from hydrochloric acid, hydrobromic acid, hydroiodic acid, phosphoric acid, sulfuric acid.

The proportions of ammonia, the temperatures and pressures of this embodiment may be as described above.

The process disclosed herein may be carried out in a way that the aqueous ammonia is used as the only solvent in the reaction. Thus, the reaction mixture does not comprise any other solvent but aqueous ammonia. This has the advantage that work-up procedures are simple. In an embodiment, compound (II) crystallises from the reaction solution in such high purity that even recrystallisation is unnecessary.

The reaction may alternatively be carried out in the presence of a solvent including n-alkanols, branched alkanols, polyols and polyethers. The solvent may be added at 10 to 50% (v/v). For example, the solvent may be added as part of a solution comprising compound (I).

The reaction may be carried out for 15 to 72 hours, 24 to 48 hours, preferably 20 to 30 hours. However, the reaction can be run up to two weeks without detrimental effect on the yield.

After completion of the reaction the crude reaction mixture may be subjected to standard work-up procedures. For example, the crude reaction mixture may be cooled to induce crystallisation, after which the product is isolated by filtration. In an embodiment, the compound (II) crystallises from the reaction solution in such high purity that recrystallisation is unnecessary. Further time and resource-consuming purification steps, such as chromatographic purification, are not necessary. Products are obtained in high purity (>99% as per HPLC measurements). Yields of 50 to 80% are obtained.

The present invention will be better understood with reference to the following examples and figures. These examples are intended to be representative of specific embodiments of the invention, and are not intended as limiting the scope of the invention.

### EXAMPLES

### Materials & Methods

The starting material, 4-hydroxy-5-methyl-(1H)-pyridin-2-one (formula (I)), is commercially available or synthesised according to the method described in Synthesis, 1984, volume 9, pages 765 to 766. The remaining agents are commercially available.

The autoclave used was a standard lab autoclave having a volume of 1000 ml or an autoclave having a volume of 50 ml.

HPLC analysis was carried out using an alkyl reversed-phase bonded phase of the type Zorbax SB-Aq (Agilent) as stationary phase, kept at 20 degrees Celsius. As mobile phase ammonium acetate buffer at a pH of 5.7 was used, which was injected during initial and final phases. Intermittently 40 to 60 vol. % acetonitrile were added to the ammonium acetate buffer at a pH of 5.7.

Yields were determined by 1H NMR using sodium tosylate as internal standard, which is completely soluble in the solution. To this end 20 mol % of sodium tosylate (based on formula (I)) were added to the reaction solution. The reaction solution was then subjected to 1H NMR measurements wherein the methyl group of formula (I) and the methyl group of the tosyl rest were integrated. In some examples yields were determined by HPLC in case of overlap in 1H NMR measurements.

### Example 1

25.0 g (0.2 mol) 4-hydroxy-5-methyl-2 (1H)-pyridin-2-one (compound I) were added to 74.9 g (1.4 mol) ammonium chloride and 225 ml (3.0 mol) ammonia water 25%. Within 4 hours the reaction mixture was heated to 170° C. in a pressure reactor under autogenous pressure (maximum autogenous pressure of 21.6 bars). After 170° C. was reached, the mixture was kept at that temperature for 24 hours and then allowed to cool to 20° C. The mixture is diluted with 36 ml water, cooled to 5° C. and kept at that temperature for 1 hour. The precipitated crystalline solid was filtered, washed with 75 ml water and dried under vacuum at 50° C. to give 19.7 g (79% yield) 4-amino-5-methyl-2 (1H)-pyridine-2-one (compound II): purity by HPLC: 100%, IR spectrum corresponds to reported IR spectrum.

A reaction following Example 1, wherein compound I was reacted with 0.5 molar equivalents of ammonium chloride at 150° C. afforded a yield of 22% (yield was determined by NMR with sodium tosylate as internal standard).

In another reaction following Example 1, compound I was reacted with 0.5 molar equivalents of ammonium chloride at 170° C. led to a yield of 50% (yield was determined by NMR with sodium tosylate as internal standard).

A reaction following Example 1, wherein compound I was reacted with 2 molar equivalents of ammonium chloride at 150° C. afforded a yield of 34% (yield was determined by NMR with sodium tosylate as internal standard).

In another reaction following Example 1, compound I was reacted with 2 molar equivalents of ammonium chloride at 170° C. led to a yield of 75% (yield was determined by NMR with sodium tosylate as internal standard).

### Example 2

125 mg (1.0 mmol) 4-hydroxy-5-methyl-2 (1H)-pyridin-2-one (compound I) were added to 225 mg (1.5 mmol) sodium iodide and 1.12 ml (15.0 mmol) ammonia water 25%. The reaction mixture was heated to 170° C. in a pressure reactor under autogenous pressure. After 170° C. was reached, the mixture was kept at that temperature for 48 hours and then allowed to cool to 20° C. Yield was determined by NMR (with sodium tosylate as internal standard) and gave 54% 4-amino-5-methyl-2 (1H)-pyridine-2-one (compound II).

A reaction following Example 2, wherein compound I was reacted with 0.5 molar equivalents of sodium iodide at 150° C. afforded a yield of 43% (yield was determined by NMR with sodium tosylate as internal standard).

In another reaction following Example 2, compound I was reacted with 0.5 molar equivalents of sodium iodide at 160° C. afforded a yield of 26% (yield was determined by NMR with sodium tosylate as internal standard).

In another reaction following Example 2, compound I was reacted with 0.5 molar equivalents of sodium iodide at 170° C. afforded a yield of 70% (yield was determined by NMR with sodium tosylate as internal standard).

A reaction following Example 2, wherein compound I was reacted with 2 molar equivalents of sodium iodide at 150° C. afforded a yield of 31% (yield was determined by NMR with sodium tosylate as internal standard).

In another reaction following Example 2, compound I was reacted with 2 molar equivalents of sodium iodide at 160° C. afforded a yield of 30% (yield was determined by NMR with sodium tosylate as internal standard).

In another reaction following Example 2, compound I was reacted with 2 molar equivalents of sodium iodide at 170° C. (maximum autogenous pressure of 20 bar) leading to a yield of 54% (yield was determined by NMR with sodium tosylate as internal standard).

### Example 3

330 mg (2.6 mmol) 4-hydroxy-5-methyl-2 (1H)-pyridin-2-one (compound I) were added to 1.29 g (13.2 mmol) ammonium bromide, 33 mg (0.1 mmol) aluminum lactate and 3.0 ml (39.8 mmol) ammonia water 25%. The reaction mixture was heated to 150° C. in a pressure reactor under autogenous pressure. After 150° C. was reached, the mixture was kept at that temperature for 72 hours and then allowed to cool to 20° C. Yield was determined by HPLC and gave 92% 4-amino-5-methyl-2 (1)-pyridine-2-one (compound II).

### Example 4

330 mg (2.6 mmol) 4-hydroxy-5-methyl-2 (1H)-pyridin-2-one (compound I) were added to 1.29 g (13.2 mmol) ammonium bromide, 33 mg (0.1 mmol) magnesium chloride and 3.0 ml (39.8 mmol) ammonia water 25%. The reaction mixture was heated to 150° C. in a pressure reactor under autogenous pressure. After 150° C. was reached, the mixture was kept at that temperature for 72 hours and then allowed to cool to 20° C. Yield was determined by HPLC and gave 91% 4-amino-5-methyl-2 (1H)-pyridine-2-one (compound II).

### Example 5

25.0 g (0.2 mol) 4-hydroxy-5-methyl-2 (1H)-pyridinone (compound I) was added to 79.3 g (0.6 mol) ammonium sulfate and 150 ml (2.0 mol) ammonia water 25%. Within 4 hours the reaction mixture was heated to 170° C. in a pressure reactor under autogenous pressure (24.0 bar). After 170° C. were reached, the mixture was kept at that temperature for 24 hours and then allowed to cool to 20° C. The mixture is diluted with 36 ml water, cooled to 5° C. and kept at that temperature for 1 hour. The precipitated crystalline solid was filtered, washed with 72 ml water and dried under vacuum at 50° C. to give 20.2 g (81% yield) 4-amino-5-methyl-2 (1H)-pyridine-2-on (compound II), purity by HPLC: 99.7%, IR spectrum compliant.

### Example 6

2.5 g (20 mmol) 4-hydroxy-5-methyl-2 (1H)-pyridinone (compound I) was added to 3.5 g (30 mmol) ammonium sulfite (NH₄)₂SO₃) and 23 ml (300 mmol) ammonia water 25%. The reaction mixture was heated to 170° C. in a pressure reactor under autogenous pressure (maximum pressure of 20 bar). After 170° C. was reached, the mixture was kept at that temperature for 48 hours and then allowed to cool to 20° C. Yield of 4-amino-5-methyl-2 (1H)-pyridine-2-on (compound II) was determined by NMR (80%).

A reaction following Example 6, wherein compound I was reacted with 0.2 molar equivalents of the sulfite at 170° C. afforded a yield of 45% determined by NMR.

In another reaction following Example 6, compound I was reacted with 1 molar equivalent of the sulfite at 170° C. affording a yield of 70% determined by NMR.

In another reaction following Example 6, compound I was reacted with 2 molar equivalent of the sulfite at 170° C. affording a yield of 85% determined by NMR.

In another reaction following Example 6, compound I was reacted with 1 molar equivalent of the sulfite at 150° C. affording a yield of 41% determined by NMR.

In another reaction following Example 6, I was reacted with 2 molar equivalents of the sulfite at 150° C. affording a yield of 70% determined by NMR.

### Example 7

330 mg (2.6 mmol) 4-hydroxy-5-methyl-2 (1H)-pyridin-2-one (compound I) were added to 13.15 mmol (5 molar equivalents) malonic acid and 3.0 ml (39.8 mmol, 15 equivalents) ammonia water 25%. The reaction mixture was heated to 170° C. in a pressure reactor under autogenous pressure. After 170° C. was reached, the mixture was kept at that temperature and reacted for 48 hours and then allowed to cool to 20° C. The yield was determined by HPLC, which lead to 87% 4-amino-5-methyl-2 (1H)-pyridine-2-one (compound II).

### Example 8

330 mg (2.6 mmol) 4-hydroxy-5-methyl-2 (1H)-pyridin-2-one (compound 1) were added to 13.15 mmol (5 molar equivalents) caproic acid and 3.0 ml (39.8 mmol, 15 equivalents) ammonia water 25%. The reaction mixture was heated to 170° C. in a pressure reactor under autogenous pressure. After 170° C. was reached, the mixture was kept at that temperature and reacted for 48 hours and then allowed to cool to 20° C. The yield was determined by HPLC, which lead to 84% 4-amino-5-methyl-2 (1H)-pyridine-2-one (compound II).

Additional examples using additives as per the table below were run as follows: 330 mg (2.6 mmol) 4-hydroxy-5-methyl-2 (1H)-pyridin-2-one (formula I) were reacted with 13.15 mmol (5 equivalents) of the additive displayed in the table and also 3.0 ml (39.8 mmol, 15 equivalents) ammonia water 25%. The reaction mixture was heated to 170° C. in a pressure reactor under autogenous pressure. After 170° C. was reached, the mixture was kept at that temperature and reacted for 48 hours and then allowed to cool to 20° C. The conversion of formula I to formula II was determined by HPLC. Additionally, the selectivity of the reaction towards formula II was determined by HPLC. Results are also displayed in the table below.

| | Conversion of | |
|---|---|---|
| | formula I | Yield |
| | measured by | determined |
| Additive | HPLC (%) | by HPLC |
| Ammonium citrate (CAS 3012-65-5) | 96.4 | 76 |
| Ammonium oxalate monohydrate (CAS 6009-70-7) | 97.4 | 80 |
| Ammonium tartrate (CAS 3164-29-2) | 94.3 | 60 |
| Tetrabutylammonium | 23.0 | 10 |
| Hydroxide | | |
| (40% wt in water) (CAS 2052-49-5) | | |
| Pyridinium bromide (CAS 18820-82-1) | 97.2 | 91 |
| Sodium tosylate (CAS 657-84-1) | 56.6 | 25 |
| Lithium triflate (CAS 33454-82-9) | 68.6 | 54 |
| Zinc chloride (CAS 7646-85-7)^{b} | 82.7 | 66 |
| Calcium carbonate (CAS 471-34-1) | 56.1 | 45 |
| Zinc acetate dihydrate (CAS 5970-45-6) | 90.6 | 79 |
| Aluminum chloride, anhydrous | 69.3 | 39 |
| (CAS 7446-70-0) | | |
| Tetrabutylammonium Chloride | 76.8 | 38 |
| (CAS 1112-67-0) | | |
| Triethylamine (CAS 121-44-8)/ | 99.8 | 25 |
| HI (55% in water) (CAS: 10034-85-2) | | |
| Magnesium chloride, anhydrous (CAS 7786-30-3) | 99.6 | 71 |
| Copper (II) sulfate, pentahydrate (CAS 7758-99-8) | 95.9 | 48 |
| Iron (II) acetate (CAS 3094-87-9) | 98.4 | 89 |

### Embodiment Section

Item 1. Process for the production of 4-amino-5-methyl-(1H)-pyridin-2-one of formula (II)

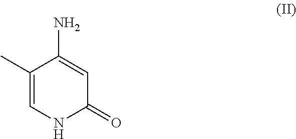

characterised in that 4-hydroxy-5-methyl-(1H)-pyridin-2-one of formula (I)

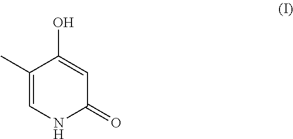

is reacted under pressure with aqueous ammonia and a salt or an acid.
Item 2. Process according to item 1, characterized in that the reaction temperature is 120 to 220° C.
Item 3. Process according to item 1 or item 2, characterized in that the reaction temperature is 150 to 190° C.
Item 4. Process according to any of items 1 to 3, characterized in that the reaction temperature is 165 to 175° C.
Item 5. Process according to any of items 1 to 4, characterized in that the salt comprises a cation selected from the group consisting of ammonium, mono-alkyl-ammonium, di-alkyl-ammonium, tri-alkyl-ammonium, tetra-alkyl-ammonium, pyridinium, and lutidinium,
and an anion selected from the group consisting of fluoride, chloride, bromide, iodide, sulfate, sulfite phosphate tribasic, carboxylates, preferably acetate, citrate, tartrate, oxalate, formate, and sulfonates, preferably mesylate, triflate and tosylate.
Item 6. Process according to item 5, characterized in that the salt is added in 0.2 to 15 molar equivalents based on formula (I), wherein the molar equivalents are calculated with respect to the ammonium cation of a salt.
Item 7. Process according to any of items 1 to 5, characterized in that the salt is selected from the group consisting of ammonium, mono-alkyl-ammonium, di-alkyl-ammonium, tri-alkyl-ammonium, or tetra-alkyl-ammonium fluoride; ammonium, mono-alkyl-ammonium, di-alkyl-ammonium, tri-alkyl-ammonium, or tetra-alkyl-ammonium chloride; ammonium, mono-alkyl-ammonium, di-alkyl-ammonium, tri-alkyl-ammonium, or tetra-alkyl-ammonium bromide; and ammonium, mono-alkyl-ammonium, di-alkyl-ammonium, tri-alkyl-ammonium, or tetra-alkyl-ammonium iodide, ammonium triflate, ammonium phosphate, ammonium acetate, ammonium citrate, ammonium oxalate, ammonium tartrate, ammonium mesylate or ammonium tosylate or pyridinium bromide.
Item 8. Process according to any of items 1 to 7, characterized in that the salt is ammonium chloride.
Item 9. Process according to item 7 or 8, characterized in that the salt is added in 5 to 10 molar equivalents based on formula (I), wherein the molar equivalents are calculated with respect to the ammonium cation of a salt.
Items 10. Process according to any of items 7 to 9, characterized in that the salt is added in an amount of 6 to 8 molar equivalents based on formula (I), wherein the molar equivalents are calculated with respect to the ammonium cation of a salt.
Item 11. Process according to any of items 1 to 5, characterized in that the salt is selected from the group consisting of ammonium, mono-alkyl-ammonium, di-alkyl-ammonium, tri-alkyl-ammonium, or tetra-alkyl-ammonium sulfate; and ammonium, mono-alkyl-ammonium, di-alkyl-ammonium, tri-alkyl-ammonium, or tetra-alkyl-ammonium sulfite.
Item 12. Process according to item 11, characterized in that the salt is added in 0.1 to 6 molar equivalents based on formula (I), wherein the molar equivalents are calculated with respect to the ammonium cation of a salt.
Item 13. Process according to any of items 1 to 12, characterized in that the salt is formed in situ by an addition of a corresponding base of the cation and a corresponding acid of the anion to provide the molar equivalents.
Item 14. Process according to any of items 1 to 4, characterized in that the salt is selected from the group consisting of alkali metal fluoride, chloride, bromide, iodide, tosylate, or triflate; and alkaline earth metal fluoride, chloride, bromide, iodide, tosylate, triflate, carbonate.
Item 15. Process according to any of Items 1 to 4 and 14, characterized in that the salt is selected from sodium iodide or potassium iodide.
Item 16. Process according to item 14 or 15, characterized in that the salt is added in an amount of 0.5 to 8 molar equivalents based on formula (I).
Item 17. Process according to any of items 1 to 16, characterized in that the reaction is carried out in the presence of a Lewis acid catalyst.
Item 18. Process according to any of items 1 to 17, characterized in that the reaction is carried out in the presence of 2 to 20 mol %, or 5 to 15 mol %, or 7 to 13 mol %, or 8 to mol 12% with regard to formula (I) of a Lewis acid catalyst.
Item 19. Process according to item 17 or 18, characterized in that the Lewis acid catalyst is selected from an inorganic or organic salt of magnesium, calcium, aluminium, boron, silicon, zinc, iron, copper, silver, titanium, and zirconium.
Item 20. Process according to any of items 17 to 19, characterized that ammonium bromide is reacted with aluminium lactate, zinc acetate, magnesium chloride or calcium chloride.
Item 21. Process according to any of items 17 to 20, characterized in that it is carried out at 130 to 170° C., 140 to 160° C., or 145 to 155° C., preferably 150° C.
Item 22. Process according to any of items 1 to 4, characterized in that the acid is an organic or inorganic acid added in an amount of 3 to 10, preferably 4 to 8 molar equivalents based on formula (I).
Item 23. Process according to item 22, characterized in that the organic acid is a carboxylix acid, preferably citric acid, oxalic acid, tartaric acid, lactic acid, malonic acid, caproic acid, formic acid, acetic acid, propionic acid, butanoic acid, isobutanoic acid, pentanoic acid, trifluoroacetic acid, or a sulfonic acid, preferably methanesulfonic acid, para-toluenesulfonic acid, trifluoromethanesulfonic acid, and the inorganic acid is preferably hydrochloric acid, hydrobromic acid, hydroiodic acid, phosphoric acid, sulfuric acid.
Item 24. Process according to any of items 1 to 23, characterized in that the aqueous ammonia comprises ammonia in an amount of 10 to 20 molar equivalents based on formula (I).
Item 25. Process according to any of items 1 to 24, characterized in that the aqueous ammonia comprises ammonia in an amount of 13 to 17 molar equivalents based on formula (I).
Item 26. Process according to any of items 1 to 25, characterized in that the aqueous ammonia comprises 14 to 16 molar equivalents of ammonia based on formula (I).
Item 27. Process according to any of items 1 to 26, characterized in that pressure is built up in an autoclave.
Item 28. Process according to any of items 1 to 26, characterized in that the reaction is carried out in a pressure reactor by applying an external pressure of 5 to 340 bar, preferably 15 to 100 bar.
Item 29. Process according to any of items 1 to 28, characterized in that the aqueous ammonia is used as an only solvent in the reaction.
Item 30. Process according to any of items 1 to 29, characterized in that a solvent is added selected from the group including n-alkanols, branched alkanols, polyols and polyethers.
Item 31. Process according to any of items 1 to 30, characterized in that the reaction is carried out for 15 to 72 hours, preferably 24 to 48 hours.

1-17. (canceled)

18. A method of producing of 4-amino-5-methyl-(1H)-pyridin-2-one of formula (II) 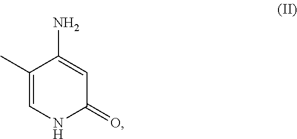
comprising reacting 4-hydroxy-5-methyl-(1H)-pyridin-2-one of formula (I) 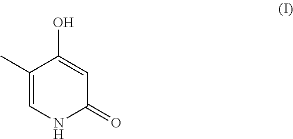
under pressure with aqueous ammonia and a salt or an acid.

19. The method according to claim 18, wherein the reaction temperature is 120 to 220° C.

20. The method according to claim 18, wherein the salt comprises a cation selected from the group consisting of ammonium, mono-alkyl-ammonium, di-alkyl-ammonium, tri-alkyl-ammonium, tetra-alkyl-ammonium, pyridinium, and lutidinium,
and an anion selected from the group consisting of fluoride, chloride, bromide, iodide, sulfate, sulfite, phosphate tribasic, carboxylate and sulfonate.

21. The method according to claim 20, wherein the salt is added in 0.2 to 15 molar equivalents based on formula (I), wherein the molar equivalents are calculated with respect to the ammonium cation of a salt.

22. The method according to claim 18, wherein the salt is selected from the group consisting of ammonium, mono-alkyl-ammonium, di-alkyl-ammonium, tri-alkyl-ammonium, or tetra-alkyl-ammonium fluoride; ammonium, mono-alkyl-ammonium, di-alkyl-ammonium, tri-alkyl-ammonium, or tetra-alkyl-ammonium chloride; ammonium, mono-alkyl-ammonium, di-alkyl-ammonium, tri-alkyl-ammonium, or tetra-alkyl-ammonium bromide; and ammonium, mono-alkyl-ammonium, di-alkyl-ammonium, tri-alkyl-ammonium, or tetra-alkyl-ammonium iodide, ammonium triflate, ammonium phosphate, ammonium acetate, ammonium citrate, ammonium oxalate, ammonium tartrate, ammonium mesylate, ammonium tosylate and pyridinium bromide.

23. The method according to claim 22, wherein the salt is added in 5 to 10 molar equivalents based on formula (I), wherein the molar equivalents are calculated with respect to the ammonium cation of salt.

24. The method according to claim 18, wherein the salt is ammonium chloride.

25. The method according to claim 24, wherein the salt is added in 5 to 10 molar equivalents based on formula (I), wherein the molar equivalents are calculated with respect to the ammonium cation of a salt.

26. The method according to claim 18, wherein the salt is selected from the group consisting of ammonium, mono-alkyl-ammonium, di-alkyl-ammonium, tri-alkyl-ammonium, or tetra-alkyl-ammonium sulfate; and ammonium, mono-alkyl-ammonium, di-alkyl-ammonium, tri-alkyl-ammonium, and tetra-alkyl-ammonium sulfite.

27. The method according to claim 18, wherein the salt is formed in situ by an addition of a corresponding base of the cation and a corresponding acid of the anion to provide the molar equivalents.

28. The method according to claim 18, wherein the salt is selected from the group consisting of alkali metal fluoride, chloride, bromide, iodide, tosylate and triflate; and alkaline earth metal fluoride, chloride, bromide, iodide, tosylate, triflate and carbonate.

29. The method according to claim 19, wherein the salt is selected from the group consisting of alkali metal fluoride, chloride, bromide, iodide, tosylate and triflate; and alkaline earth metal fluoride, chloride, bromide, iodide, tosylate, triflate and carbonate.

30. The method according to claim 18, wherein the reaction is carried out in the presence of 2 to 20 mol %, with regard to formula (I) of a Lewis acid catalyst.

31. The method according to claim 18, wherein the acid is an organic or inorganic acid added in an amount of 3 to 10 molar equivalents based on formula (I).

32. The method according to claim 18, wherein the aqueous ammonia comprises ammonia in an amount of 10 to 20 molar equivalents based on formula (I).

33. The method according to claim 18, wherein pressure is built up in an autoclave.

34. The method according to claim 18, wherein the reaction is carried out in a pressure reactor by applying an external pressure of 5 to 340 bar.

35. The method according to claim 18, wherein the aqueous ammonia is used as an only solvent in the reaction.

36. The method according to claim 18, wherein the reaction is carried out for 15 to 72 hours.
